(19) United States
(12) Patent Application Publication
(10) Pub. No.: US 2024/0016522 A1
(43) Pub. Date: Jan. 18, 2024

(54) DUAL COMPRESSION INTRAMEDULLARY NAIL SYSTEM AND METHOD FOR USE IN A TALAR TIBIA CALCANEAL FUSION

(71) Applicant: ORTHOSOLSOLUTIONSHOLDINGSLIMITED, Lakewood, CO (US)

(72) Inventors: Kevin STAMP, Chapeltown Sheffield (GB); Dustin DUCHARME, Littleton, CO (US); David N. Garras, Naperville, IL (US); Justin Michael KANE, Dallas, TX (US); Andrew LEITHER, Akron, OH (US)

(21) Appl. No.: 18/350,995

(22) Filed: Jul. 12, 2023

Related U.S. Application Data
(60) Provisional application No. 63/388,372, filed on Jul. 12, 2022.

(51) Int. Cl. A61B 17/72; A61B 17/17

(52) U.S. Cl. CPC A61B 17/7225; A61B 17/7241; A61B 17/1725; A61B 17/7291

(57) ABSTRACT

The present invention provides an intramedullary nail implant system in which the nail body includes drive components which provide for independent compression on medial lateral screws to compress the interface between the tibia and talus, and in the sub-talar joint between the talus and calcaneus. An outrigger is provided which functions as a drill guide and helps to stabilize the construct and create and hold compression during the surgical procedure and a surgical method is provided which uses the system to achieve ankle fusion.

### FIELD OF THE INVENTION

The present invention relates generally to the field of orthopedics implants and surgery, and more particularly to an intramedullary implant which achieves multiple levels of compression in an associated joint bone or bone complex and a method of its use which can be used to secure fusion across multiple joints, such as the ankle joint.

### BACKGROUND OF THE INVENTION

An important category of human bones is known as the long bones, and includes the femur, fibula, tibia, humerus, radius, and ulna. These bones are frequently subjected to trauma, such as from accidents, which results in fractures that are complex and difficult to treat. For example, the femur and the tibia, are frequently fractured into several segments and must be realigned in order to facilitate normal function following healing. Mechanical hardware, such as pins, plates, screws, nails, wires and external devices are commonly used to treat fractured long bones and they are frequently made of a durable material compatible to the human body, for example titanium, stainless steel or cobalt chromium. One orthopedic implant for such use is intramedullary nails that are positioned in the intramedullary canal of those portions of the fractured bone.

Prior art devices used to treat these fractures have included means to induce compression in the associated bone in order to facilitate fusion of the bone segments. This includes fusion across a fracture line or a joint in bone segments that have been properly prepared. Fractures of the neck, head or intertrochanter of the femur have been successfully treated with a variety of compression screw assemblies, which includes a compression plate having a barrel member, a lag screw and a compressing screw. The compression plate is secured to the exterior of the femur and the barrel member is inserted into a predrilled hole in the direction of the femoral head. The lag screw which has a threaded end and a smooth portion is inserted through the barrel member so that it extends across the break and into the femoral head. The threaded portion engages the femoral head. The compressing screw connects the lag screw to the plate. By adjusting the tension of the compressing screw, the compression (or “reduction”) of the fracture can be adjusted. The smooth portion of the lag screw must be free to slide through the barrel member to permit the adjustment of the compression screw.

Femoral shaft fractures have also been treated with the help of intramedullary rods or “nails” inserted into the marrow canal of the femur to immobilize the femur parts involved in fractures. A single angled cross-nail or locking screw is inserted through the femur and the proximal end of the intramedullary rod. In some varieties, one or two screws may also be inserted through the femoral shaft and through the distal end of the intramedullary rod. The second screw may serve the purpose of preventing rotation of the implant or of the associated bone.

Intramedullary implants have also found use in repair of other bones, including other long bones, and small bones. More recently, they have also been used to treat degenerative conditions, such as osteoarthritis, through the fusion of the bones which comprise the affected joint.

The ankle joint, comprised of the tibiotalar and subtalar joints, is particularly prone to arthritic conditions which result in patient pain and difficulty in daily activities. These conditions are usually the result of wear, especially when associated with mis-alignment of other bones of the foot, leg or spine; congenital conditions or disease, such as rheumatoid arthritis; or previous trauma. In the event that less invasive treatments, such as physical therapy, fail to provide relief, ankle fusion is one method of surgical treatment. This involves the implantation of hardware, including screws, pins, rods, and plates, and associated instruments and jigs to allow for the proper placement within the joint.

The present invention provides a unique solution to fusion of the ankle joint, which include an implant spanning a variety of interacting bones, and in particular, the talus, tibia, and the calcaneus. Moreover, this solution provides an implant that provides for independent compression between the tibia and talus, and in the sub-talar joint between the talus and calcaneus. An outrigger is provided which functions as a drill guide and helps to stabilize the construct and create and hold compression during the surgical procedure.

### SUMMARY OF THE INVENTION

The invention relates to an intramedullary nail having associated screws which extends through the nail at an angle, i.e. from 60° to 90°, and preferably 90°+/−10° or 5°, to the nail long axis to hold the nail in the intramedullary canal. Specifically, these nails include a first proximal most medial/lateral anchor screw extending through the nail into the tibia which anchors the implant in the tibia, a first medial/lateral compression screw placed more distally than the anchor screw and which extends through the nail in a elongated compression slot to hold the nail relative to the talus and create compression between the talus and the tibia, and a second medial/lateral compression screw which extends through a second and more distal elongated compression slot in the nail to hold the nail relative to the calcaneus, and to create compression between the calcaneus and the talus. An additional nail and yet more distal screw may reside in the calcaneus and help to secure the nail and screws in position in the ankle and lock the construct into the dual compression configuration.

In a first embodiment, compression is achieved at the first compression screw by means of an internal drive insert assembly having a cannulated externally threaded drive sleeve that mates with internal threads in a central chamber of the nail body. This drive sleeve includes a torque driving recess, such as a hex so that the drive assembly can be driven by an associated first compression screwdriver. The opposite end includes a flange or series of flanges that secure a drive cap to the drive insert, but allows it to rotate and to ride freely relative to the internal threads of the central chamber. The drive cap flares upward to form a cradle for the peripheral threads of an associated compression screw. This drive assembly locks the first compression screw in the far end of the compression slot and is held in position by the threaded connection of the drive sleeve and the central chamber. This acts to provide proximal compression in the direction of the central long axis of the cylindrical shaped nail against a first transverse compression screw so as to push the screw proximally and create compression between the tibiotalar joint.

Compression is achieved at the second compression screw by means of a second compression screwdriver with an externally threaded shaft which is journaled in an elongated draw bolt insert or sleeve within the inner chamber of the stage portion of an outrigger at the distal end of the nail. This second compression screwdriver mates with threads on the inner surface of a recess within the draw bolt at a first end of the draw bolt. External annular bearing surfaces on the sleeve help to maintain the co-axial alignment of the inner threaded surface of the sleeve and the central long axis of the nail. This specially threaded draw bolt sleeve mates with the external threads of the second compression screwdriver which acts to advance the screwdriver shaft within the central chamber of the nail body and press against a second transverse compression screw in the proximal direction as it is tightened in the nail.

In the first embodiment, the stage portion of the outrigger contains an externally threaded surface at its proximal end for an additional mode compression along the long axis of the nail body. The stage portion of the outrigger is keyed to the implant and maintains alignment of the nail body with the draw bolt and the rest of the outrigger. An externally threaded surface on the distal end of the stage is mated with an internally threaded compression nut that has a compression surface perpendicular to the long axis of the nail body. As the compression nut is rotated relative to the stage the compression surface is allowed to press against the bottom of the foot to apply pressure to the bones and compress the joints around the nail body.

Maintenance of the compression is further ensured by the insertion of a calcaneal locking screw and an end cap which holds the stage portion of the outrigger against movement when the construct is fully implanted. Thus, the nail of the present invention can, in the first embodiment create independent compression over two joints.

In a system for the implantation of the nail, an outrigger has a stage member that mates onto the end of the nail body. The stage has a central chamber that contains the draw bolt and a proximal alignment face with a protrusion that is keyed to a notch in the distal end of the implant. As the draw bolt is screwed into the nail body axial and rotational alignment of the implant body is maintained between the implant body and an L-shaped extension where the long leg of the L extends in the direction of the nail axis and includes through holes to act as a drill guide for the placement of the cross screws. The short arm of the outrigger has a collar that surrounds the distal end of the axial portion of the stage which has position detents to indicate the medial/lateral and anterior/posterior radial positions of the L-shaped extension so as to properly align pilot holes relative to the anterior/posterior holes in the nail. In one embodiment external threads on the stage extend distally through the collar and mate with a knob. The knob is connected to the collar with bearings that allow the knob to rotate freely about the collar and acts to allow the stage to be unseated from position detents in the collar and for the collar and L-shaped extension to be rotated from one detent position to another. Thus, the short leg of the L not only provides for an offset of the drill guide relative to the nail (to provide room for the leg), but also includes detents that lock into a recess relative to the stage of the outrigger to keep it from rotating within the bone. In addition, the outrigger contains a strike plate component that is in direct contact with the distal end of the collar such that when the most distal face is struck, as when seating the implant into bone, the forces are transferred from the strike plate to the collar and avoids striking directly on the stage or draw-bolt.

In a second embodiment, compression is still created at both the tibia/talar joint and the talar/calcaneal joints, but rather than the threaded internal sleeve design of the first design, the sleeve comprises a first body member and a mating second body member which have a contractable relationship along the first axis to allow the distance between opposing compression holes in each body member to be “retracted” or shortened by means of a canulated internal threaded drive which has an internal drive hex to receive the torque driving member of a screwdriver. The internal threaded drive has a right hand thread at a first end and a left hand thread at a second end which acts mechanically like a turnbuckle insert to shorten the length of the two component body and drive compression at a talar compression screw and a calcaneal compression screw. The components have a mating configuration which parts longitudinally to allow the assembled body to compress in that direction.

The second embodiment also utilizes the outrigger to hold the construct in a stable position prior to the insertion of the locking screw and end cap which ultimately holds the axial compression and inhibits rotation.

The invention also relates to a method of achieving fusion in the ankle by providing means to achieve compression at both the tibial/talar joint and at the talar/calcaneal joints. The method involves a surgery in which an intramedullary nail is inserted through the calcaneus, the talus, and into the tibia, and an outrigger is used which includes a drill guide for pilot holes for cross holes, and also holds the nail to inhibit rotation. Further the method includes the implantation of transverse nails through the cross holes in the nail into the bone, beginning proximally in the tibia, and including having a first compression hole with a nail in the talus, and a second compression hole with a nail in the calcaneus, and having at least a final distal calcaneal nail to hold the compression at the tibiotalar joint and the talocalcaneal joints. The outrigger is removed after placement of the screws, and an end cap is added to the nail to maintain compression and close the nail.

### BRIEF DESCRIPTION OF THE DRAWINGS

FIG. 1 is a bottom end view of an intramedullary nail system with a transverse screw and outrigger where the nail is provided in two embodiments;

FIG. 2 is a top side view of a cross-section of the intramedullary nail system of FIG. 1 with an externally threaded screwdriver to provide compression against a transverse compression screw;

FIG. 3 is a bottom end view of the intramedullary nail system of FIG. 1 with a screwdriver in position to drive compression to the second compression screw;

FIG. 3A is a cross sectional view taken through the medial plane of the intermedullary nail system shown in FIG. 3;

FIG. 3B is a cross sectional view of an alternative embodiment of the intramedullary nail system of the present invention;

FIG. 4 is a side view of a cross-section of the first drive insert assembly;

FIG. 5 is a side view of the intramedullary nail system of FIG. 1 showing the first step to fix the nail and achieve compression in an ankle for an ankle fusion surgery;

FIG. 6 is a side view of the intramedullary nail system in the ankle fusion surgery shown in FIG. 5 showing the second step to achieve compression;

FIG. 7 is a side view in cross-section of the intramedullary nail system in the ankle fusion surgery shown in FIG. 5 showing the third step to achieve compression;

FIG. 7A is a bottom side view of the intramedullary nail system as shown in FIG. 7;

FIG. 8 is a side view of the intramedullary nail system in the ankle fusion surgery shown in FIG. 5 illustrating the outrigger;

FIG. 9 is a side view of the intramedullary nail system in the ankle fusion surgery shown in FIG. 5 showing the fourth step to achieve compression;

FIG. 10 is a side view in cross section of the intramedullary nail system in the ankle fusion surgery shown in FIG. 5 showing the fourth step to achieve compression;

FIG. 10A is a bottom side view of the intramedullary nail system as shown in FIG. 10;

FIG. 11 is a side view which is similar to FIG. 10, but showing more detail of the outrigger and screwdriver;

FIG. 12 is a side view of the intramedullary nail system in the ankle fusion surgery shown in FIG. 5 showing the sixth step to achieve compression;

FIG. 13 is a side view of the intramedullary nail system in the ankle fusion surgery shown in FIG. 5 showing the seventh step to achieve compression;

FIG. 14 is a side view of the intramedullary nail system in the ankle fusion surgery shown in FIG. 5 showing the eighth step to achieve compression;

FIG. 15 is a side view of a second embodiment of the intramedullary nail of the present invention;

FIG. 16 is a side view of a cross-section of the embodiment of the intramedullary nail of FIG. 15; and

FIG. 17 is a side view of the cross-section of the intramedullary nail system of FIG. 16 showing additional detail of the compression feature.

### DETAILED DESCRIPTION OF THE DRAWING

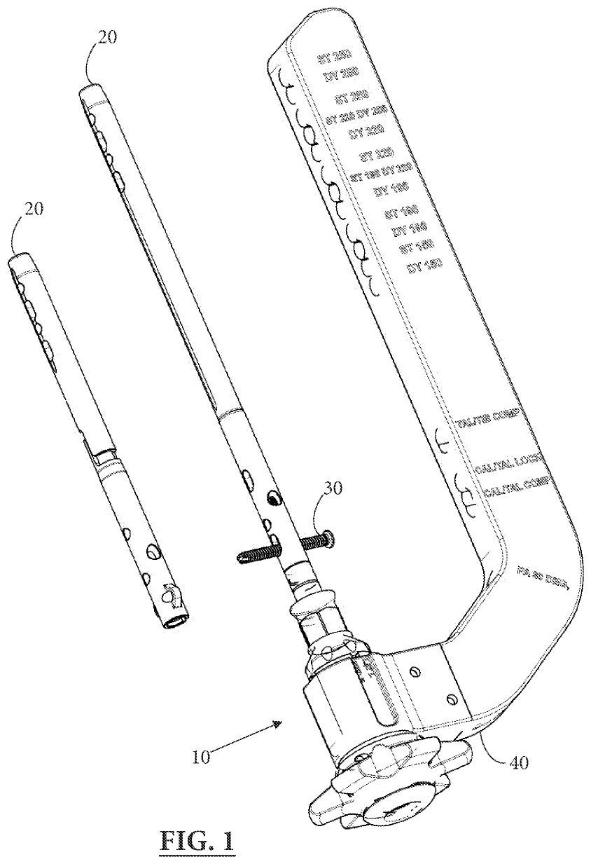

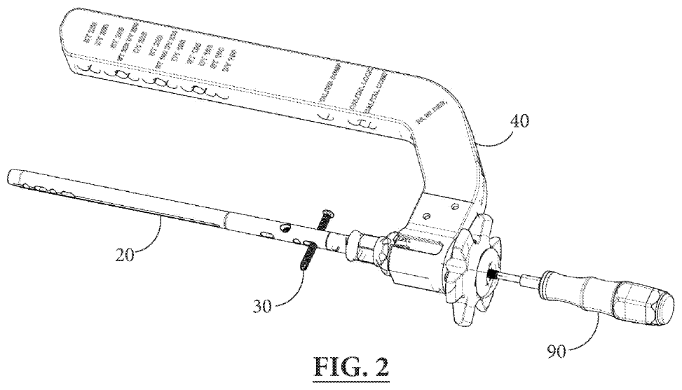

The present invention provides an intramedullary nail specifically designed for fusion of the TTC (Tibia/Talo/Calcaneal) joints. In particular, this is achieved by providing an implant system 10 shown in a first embodiment in FIG. 1 comprising the implant 20, screws 30, and an outrigger 40 and method of surgery that allows for independent compression at both of the tibia/talar interface and at the talar/calcaneal interface.

The nail 20 has a body member 22 which is an elongated multidiameter hollow outer cylinder having at least one external conical ramped area 25 transitioning between a smaller distal diameter from a greater proximal diameter that act to help provide additional compression in the direction of the long axis of the cylinder body. Along the long axis, the cylinder has a hollow center chamber 24 that includes internal threads 125 along at least a portion of the chamber.

The chamber 24 receives an internal drive insert assembly 100, 101 which includes the drive insert having a torque recess and external threads 105 that mate with chamber internal threads 106 to help to apply an axial force to the associated compression screw 30. The drive insert assembly includes an internal hex to receive an associated screwdriver and at the opposite end includes a cap member 101 that clips onto flanges 103 so as to allow the cap to float relative to the internal threads of the central chamber.

The second drive is created by a screwdriver which has a threaded engagement in a draw bolt sleeve 120 that has internal threads 124 that mate with external threads 125 on the screwdriver The proximal most screw is an anchor screw in the tibia and the more distal and longer drive insert 120 is used to drive compression at both of the compression holes. The draw bolt sleeve 120 resides within a portion of the outrigger 40 and acts against it as the screwdriver drives compression to compress the nail system.

The compression screws extend perpendicular to the long axis of the body member through medial/lateral through holes 80 in the nail 22. From distal to proximal, the nail includes several through holes for at least these screws as follows: a tibial anchor 32, a first compression screw 32 which provides an axial force at the tibia talar interface, a second compression screw 34 which provides an axial force at the tibia calcaneus interface, and a final locking screw 38 in the calcaneus, which is placed after compression is achieved in the joints to maintain the compressed alignment of the bones. The nail also includes one or more anterior posterior 39 through holes with screws to help to keep the radial position of the nail.

During the surgical procedure, an outrigger 40 has an outrigger mechanism 50 to help to hold the compression and to avoid radial rotation about the long axis through the application of an external force by a knob 48 which draws the stage 52 into detents 60 in the collar 410 which mate with protrusions 401 on the stage to prevent rotation. After the placement of the locking screw 38, this aid is not necessary and is removed. A cap (not shown) is placed at the heel end of the internal chamber 24 to hold the internal drive inserts 100,120 in position and assure that the proximal insert 120 does not work its way through the nail 20 to protrude at the calcaneus.

The drive inserts 100, 120 include drive recesses 101, 121 and a screwdriver 90 is used to drive the inserts.

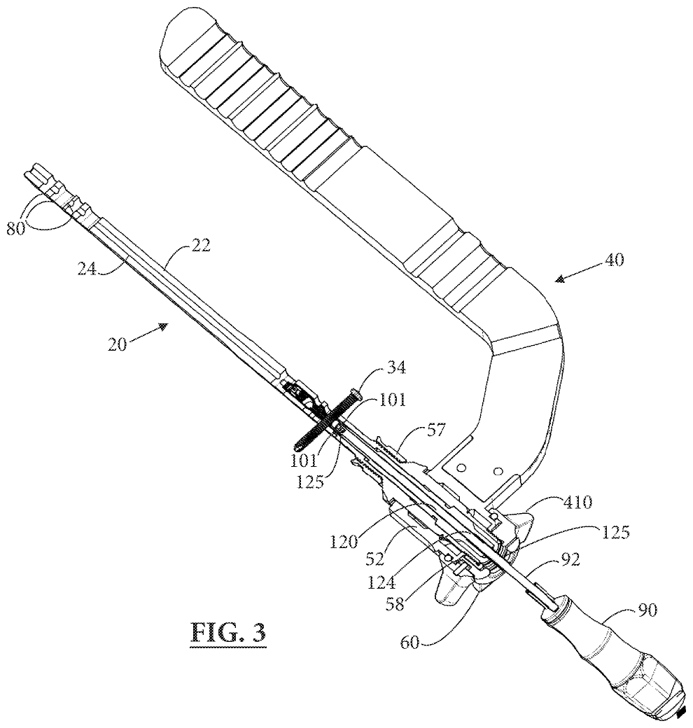

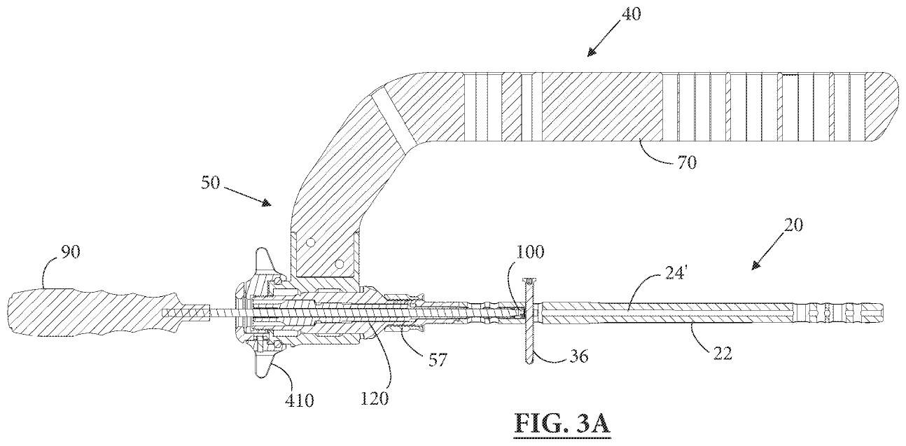

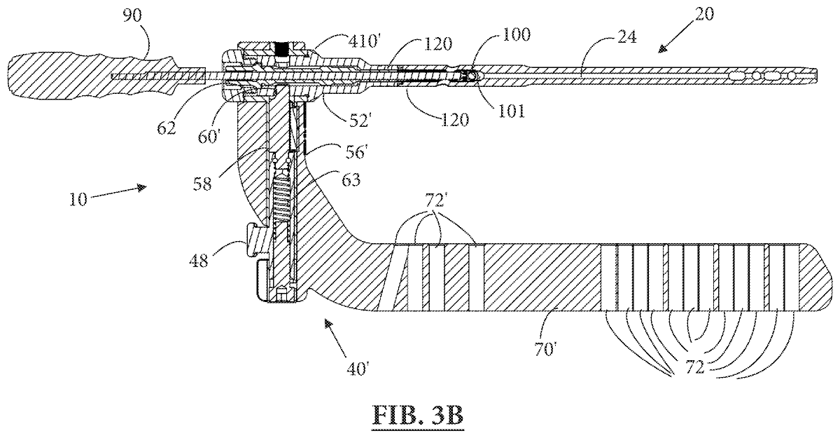

The outrigger 40 has an annular stage 52 which includes a portion that is an alignment face with a protrusion that is keyed into a notch on the end proximal most (relative to the instrument and not the anatomy) end of the nail 20. An annular ring member 55 has an indicia 53 to show the radial position of the outrigger 40. The outrigger has an L-shaped configuration with a short arm 56 and a long arm 70 that has guide holes 72 placed to guide the drilling of pilot holes or for the direct placement of fixation means through the through holes 80 in the nail 20. In the embodiment shown in FIG. 3B, the short arm 56′ of the outrigger includes an internal recess 58 that houses the spring 63 to bias the detent 62 toward the nail in an orientation transverse to the long axis of the nail.

A screwdriver 90 is used to drive the drive inserts so as to achieve compression in the compression slot screw holes including the proximal compression hole 84 and the distal compression hole 86. The driver has a shaft member 92 includes external threads 96 that mates with internal threads 97 on the insert 120. The shaft 92 has a tip 94 that bears against the compression screw 30 to drive the screw in the compression slot and to cause compression at the corresponding joint.

In order to apply compression to the more distal compression screw, the screwdriver 90 is rotated in relation to the insert draw bolt component 120 within the portion 51 of the outrigger attached to the end of the nail 22 and connected to the outrigger stage 52. This section of the stage has an opening which is coaxial with the central chamber 24 of the nail, and which receives the draw bolt component insert 120 which acts along with the associated anchoring screw or screws as a tension device to draw the nail and outrigger assembly together. This insert 120 includes internal threads that mate with external threads on the screwdriver 90 and also includes an annular shoulder with a surface facing toward the proximal end of the nail assembly. This surface opposes a mating surface on the outrigger stage member 51 so that when the screwdriver advances through the threaded engagement with the draw bolt component, the draw bolt insert 120 acts as a tension coupler to apply pressure to the stage component proximally in the direction of the long axis of the central chamber. In addition, the outer surface of the outrigger portion 51 includes a ramped area of greater diameter that also bears against the associated bone when the screwdriver is rotated. The internal surfaces of the opening of the stage component and the external surfaces of the draw bolt insert 51 have annular bearing surfaces to maintain the axial alignment during the application of this pressure. The force is transferred to the compression screw 30 through the screwdriver 90 since the nail 22 and outrigger assembly are locked in position by the tibia screw, and the calcaneal compression screw translates the force to the calcaneus which results in compression in the sub-talar joint. An additional thumb driven locking collar 57 is provided for additional compression to avoid looseness where it is indicated.

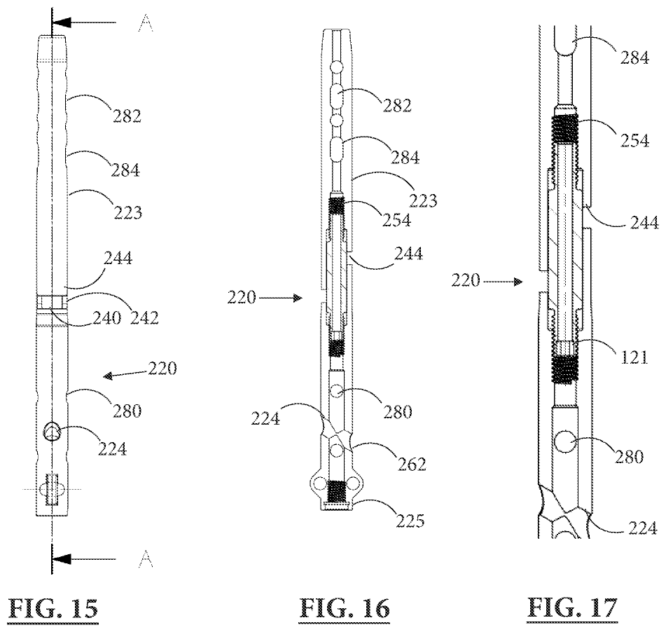

FIG. 15-17 illustrate a further embodiment of the IM nail 220 which is provided as a two-part body assembly 223. The first and second members have a mating relationship 240 with mating longitudinal parting members 242, 244 which allow for a translation of the members relative to each other to change the length. Again, the nail includes compression holes 224,280, 284 and an internal drive insert 254 which is threaded at either end to engage internal threads on the drive body and drive the retraction or extension of the body assembly and to cause compression in the associated compression screws. The end 225 of the lower member includes threads 262 for an end cap.

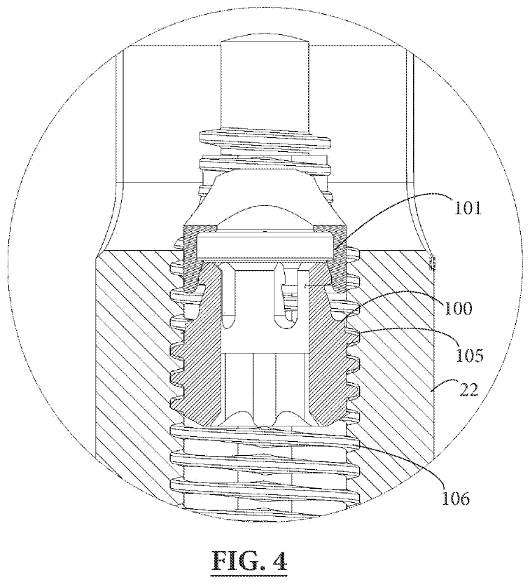

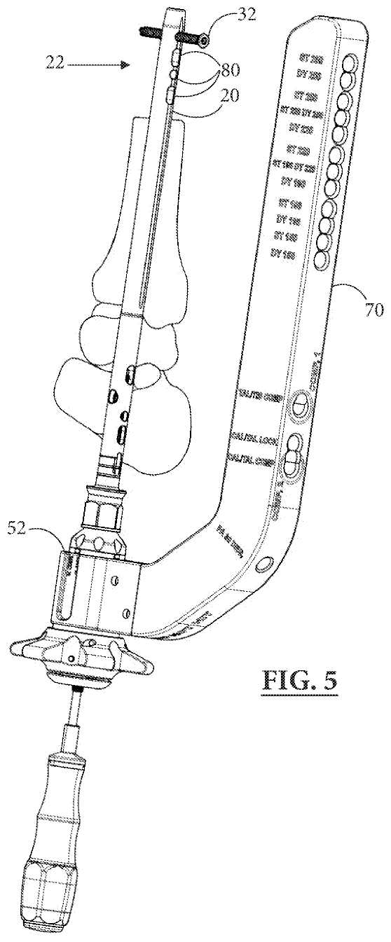

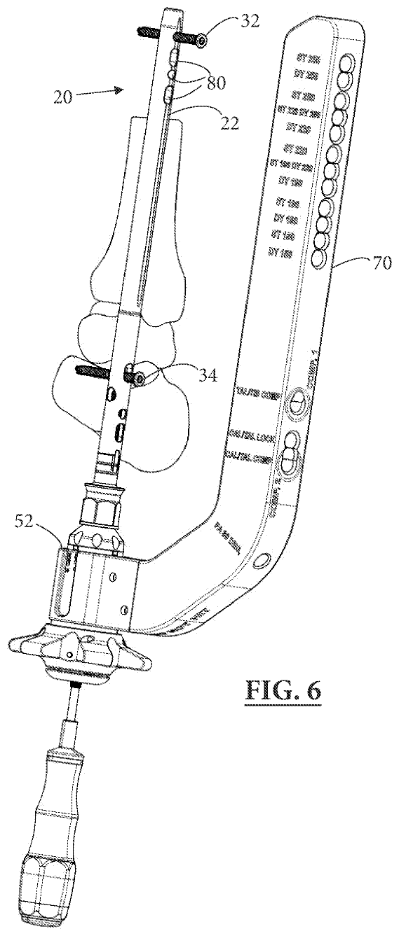

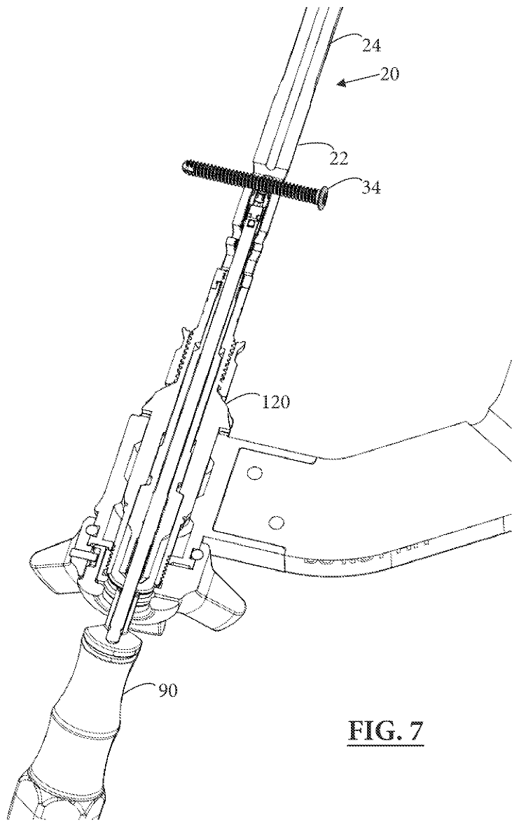

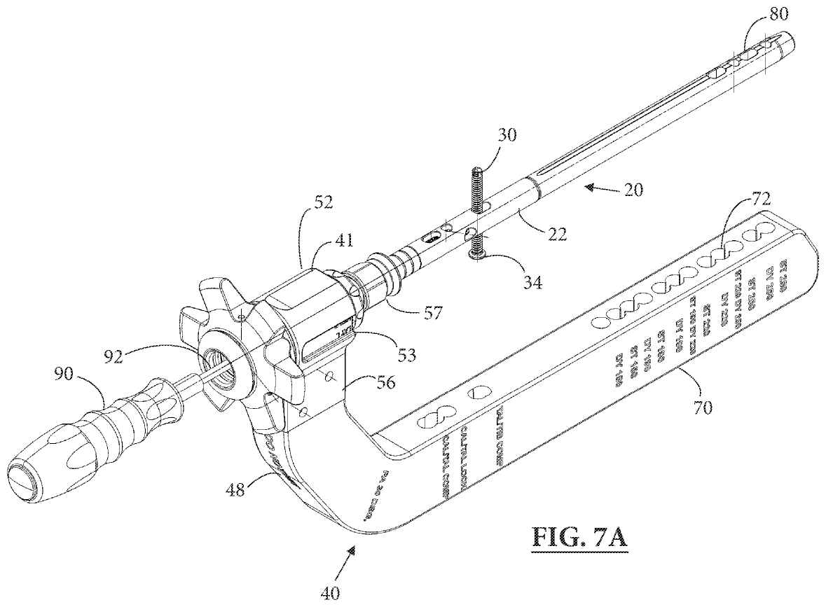

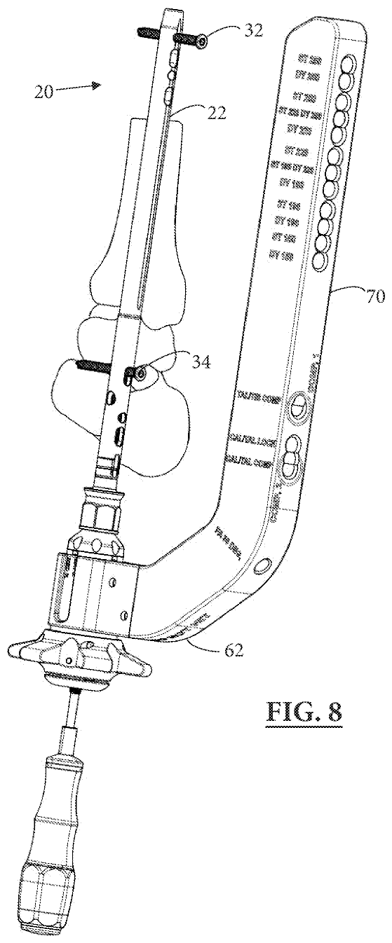

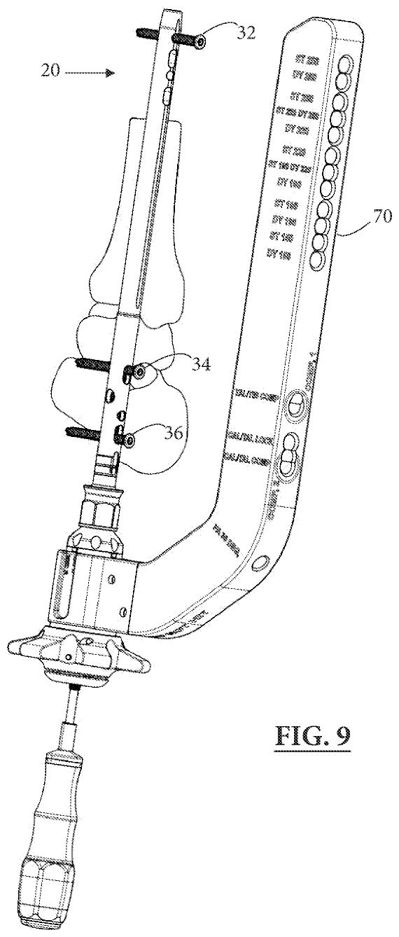

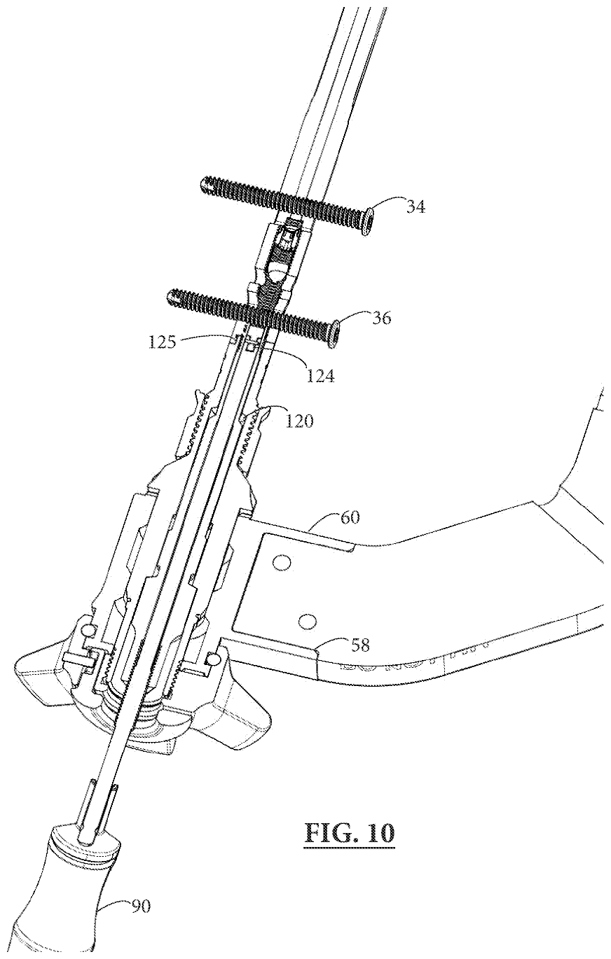

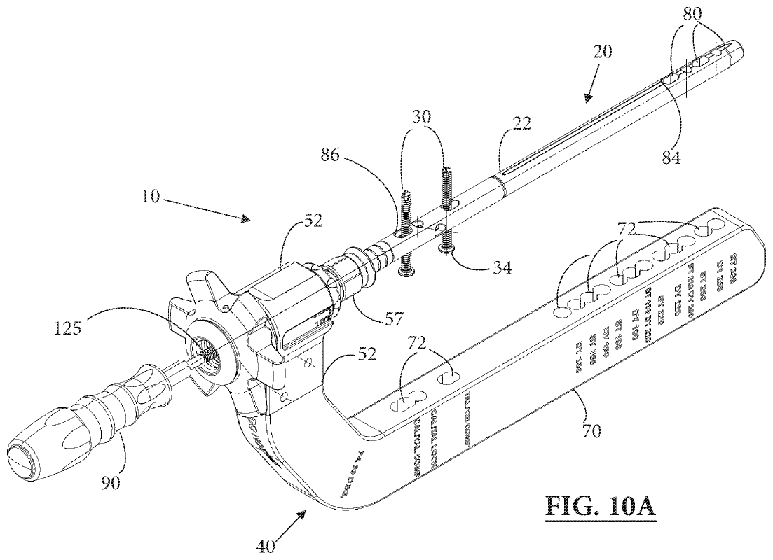

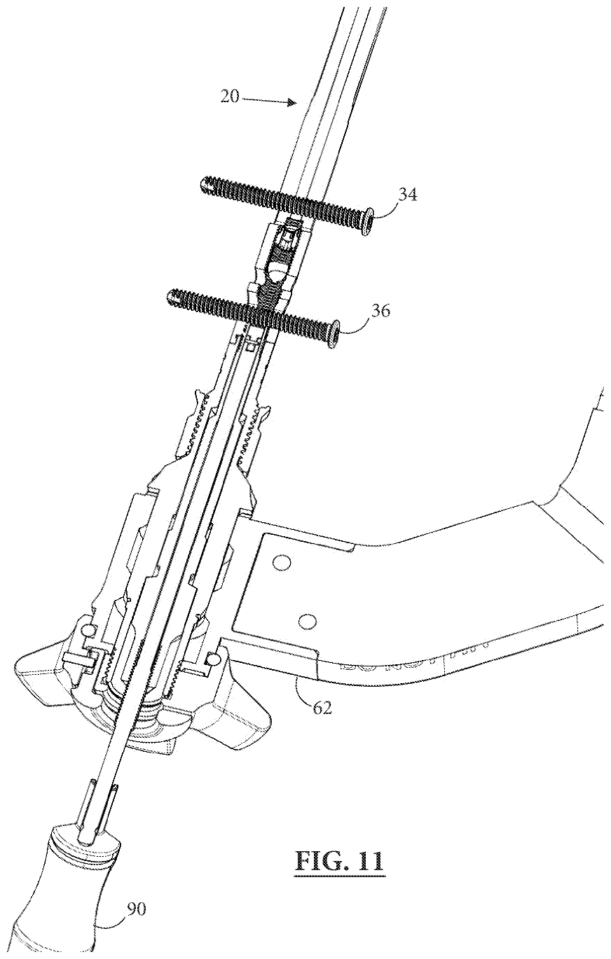

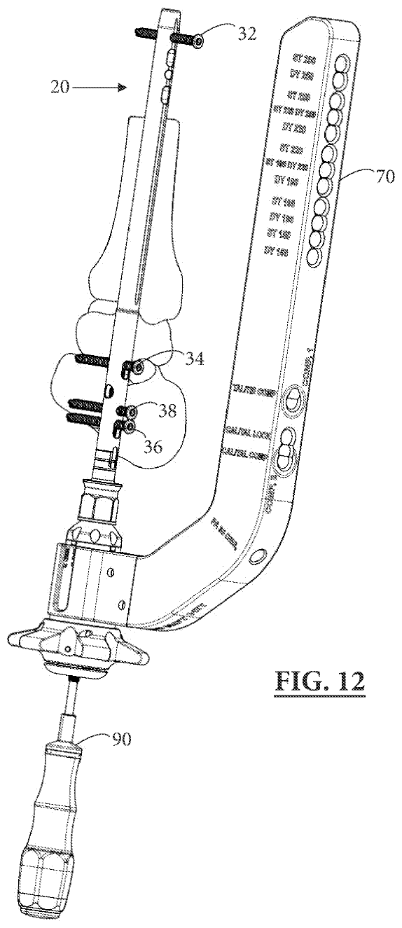

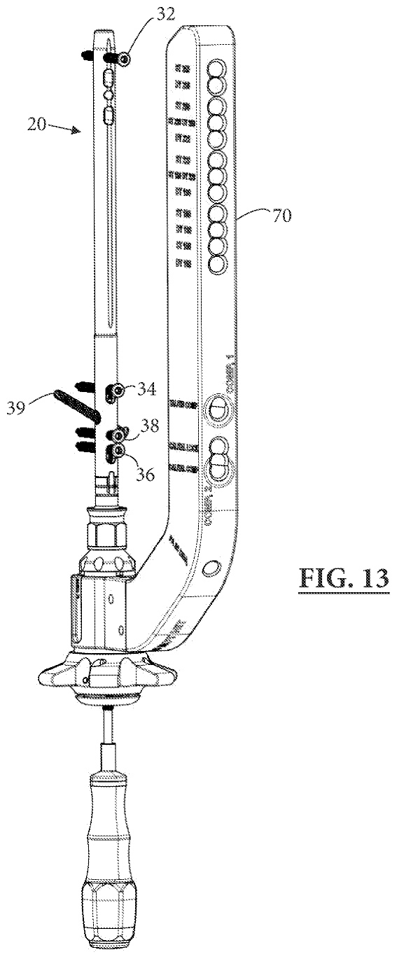

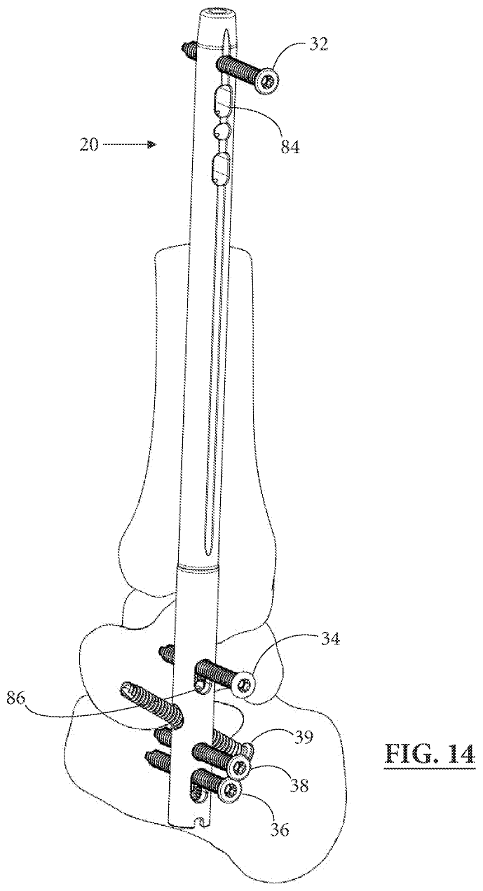

In a surgical method which is illustrated in FIGS. 3-12 for the fusion of the tibial/talar/calcaneal joint, (“the TTC joint”), using the first embodiment of the intramedullary (“IM) nail (of the present invention is described as follows:

The area is prepped and a portion of the tibia, talas, and calcaneus are reamed to receive the IM nail. The outrigger of the present invention is assembled with the end of the nail which includes an end portion that screws into the nail and a stage and L-shaped double legged member to hold against rotation and provide a drill guide.

FIG. 3 then illustrates a step in which a proximal screw (relative to the patient leg) is placed in a lateral/medial orientation through a proximal screw hole in the nail to secure the nail in the tibia. The first insert is tightened to be sure to created compression in this through hole. Care is taken to ensure that 10 mm of compression can be achieved without the distal aspect of the nail being too prominent. In the second step, the talar screw is positioned in the talar compression slot. In the third step, the screwdriver is used to tighten the adjuster insert that pushes against this medial lateral screw to create compression between the tibia and talar joints. This causes the talus screw to push the talar joint toward the distal tibia, creating compression between the two joints. In a fourth step, the distal calcaneal screw is inserted into the distal part of the calcaneal nail slot. In a fifth step, the specially threaded shaft of the drive insert is used to tighten the construct to now achieve compression between the calcaneus and sub-talus. The shaft is kept in position through the completion of the next step in order to avoid the loss of compression. Next, in the sixth step, a screw is inserted into the next hole up from the calcaneal compression slot to keep the calcaneus and talus compressed, and the threaded drive insert is now removed from the nail body. In a seventh step, a posterior anterior screw is inserted through the calcaneus and through the nail, along with any other additional screws deemed advisable. In the eighth step, the outrigger is removed and an end cap is inserted which pinches against the distal most calcaneal screw in the slot to further stabilize the construct. The incision is closed.

In accordance with the patent statutes, the best mode and preferred embodiment have been set forth; the scope of the invention is not limited thereto, but rather by the scope of the attached claims.

What is claimed is:

1. An intramedullary nail system comprising an intramedullary nail and at least three associated screws, wherein the nail includes an elongated nail body member having a central chamber extending along a long axis having internal threads and at least three through holes comprising a distal screw hole, an intermediate screw hole and a proximal compression hole, each of which extend at an angle to the long axis, wherein two of the through holes are compression holes which have a cross sectional configuration taken along the axis of the screws having a dimension that is greater than the mating diameter of an associated screw so as to form an ovoid compression hole, and the system includes one drive insert which has external threads and which is positioned in the central chamber such that the drive insert can be rotated to drive an associated screw in the proximal compression hole.

2. An intramedullary nail system as set forth in claim 1, wherein the drive insert comprises an assembly having a sleeve with a torque driving recess and external threads that mates with internal threads within the central chamber and a cap that does not engage the threads and which engages the compression screw.

3. An intramedullary nail system as set forth in claim 2, further including a screwdriver which extends into the central chamber for use and which has a threaded shaft that causes compression against a second compression screw.

4. An intramedullary nail system as set forth in claim 1, wherein at least one through hole is a distal hole along the long axis and has a round cross-sectional configuration and has an associated screw to anchor the nail body member in the associated bone.

5. An intramedullary nail system which has a nail which extends a long an axis and having a body member which has a first component which mates along a parting line with a second component in a mating configuration that permits the length of the mating configuration to be reduced to retract the length of the body member along the axis and create compression in an associated compression screw and wherein the first component and the second component together define a central chamber which includes internal threads a drive insert that has a set of external threads that are right handed and a set of external threads that are left handed and the external threads of the drive insert mate with internal threads of the central chamber so that the drive insert acts to retract the body member.

6. An intramedullary nail system as set forth in claim 1, further including an outrigger which is external to but connected to the nail during surgery to provide a drill guide to placement of the screws.

7. An intramedullary nail system which has a nail which extends a long an axis and having a body member which has a first component which mates along a parting line with a second component in a mating configuration that permits the length of the mating configuration to be reduced to retract the length of the body member along the axis and create compression in an associated compression screw including a threaded screwdriver, an outrigger which is external to but connected to the nail during surgery to provide a drill guide to placement of the screws, and wherein the outrigger has an inner recess which is co-axial with the long axis of the central chamber, and the inner recess includes internal threads which are engaged by the threaded screwdriver to compress the system.

8. An intramedullary nail system as set forth in claim 7, wherein the outrigger also includes a detent to hold at least one component of the nail system in a desired position during surgery.

9. An intramedullary nail system as set forth in claim 8, wherein the detent retains the nail body from rotation when the drive insert is driven to create compression.

10. An intramedullary nail system comprising an intramedullary nail and at least three associated screws, wherein the nail includes an elongated two component nail body member having a central chamber extending along a long axis having internal threads and at least three through holes extending at an angle to the long axis, wherein two of the through holes have a cross sectional configuration taken along the axis of the screws having a dimension that is greater than the mating diameter of an associated screw so as to form an ovoid compression hole, and the system includes a drive insert which has a set of right hand external threads and a set of left hand external threads and which engaged in the central chamber such that the drive insert can be rotated to drive an associated screw in each of the compression holes and to retract the nail body member components along the long axis.

11. An intramedullary nail system comprising an intramedullary nail and at least three associated screws and an outrigger, wherein the nail includes an elongated nail body member having a central chamber extending along a long axis having internal threads and at least three through holes extending at an angle to the long axis, wherein two of the through holes have a cross sectional configuration taken along the axis of the screws having a dimension that is greater than the mating diameter of an associated screw so as to form an ovoid compression hole, and the system includes one or more drive insert which has external threads and which is engaged in the central chamber such that the drive insert can be rotated to drive an associated screw in each of the compression holes and the outrigger which serves as a drill guide during surgery to implant the nail and screws.

12. An intramedullary nail system as set forth in claim 11, further including a threaded screwdriver and wherein the outrigger houses a draw bolt sleeve having an inner recess that is coaxial with the central chamber and the draw bolt sleeve includes internal threads that receive the external threads of a screwdriver to compress the nail system and draw the nail and compression screws into compression across two compression slots.

13. A method of surgery for fusion of the tibiotalo and talocalcaneal joints which comprises implanting an intramedullary nail in the tibia talus and calcaneus, the nail comprising an intramedullary nail and at least three associated screws, wherein the nail includes an elongated nail body member having a central chamber extending along a long axis having internal threads and at least three through holes extending at an angle to the long axis, wherein two of the through holes have a cross sectional configuration taken along the axis of the screws having a dimension that is greater than the mating diameter of an associated screw so as to form an ovoid compression hole, and a first screw is inserted through hole into the tibia to form an anchor, and a second screw is inserted into a compression hole in the talus and a third screw is inserted into a compression hole in the calcaneus and compression is driven by on the screws in the direction of the long axis to compress a bone interface at each of the tibiotalar joint and the talocalcaneal joint.

14. A method of surgery for fusion of the tibiotalo and talocalcaneal joints which comprises implanting an intramedullary nail in the tibia talus and calcaneus, the nail comprising an intramedullary nail and at least three associated screws, wherein the nail includes an elongated nail body member having a central chamber extending along a long axis having internal threads and at least three through holes extending at an angle to the long axis, wherein two of the through holes have a cross sectional configuration taken along the axis of the screws having a dimension that is greater than the mating diameter of an associated screw so as to form an ovoid compression hole, and a first screw is inserted through hole into the tibia to form an anchor, and a second screw is inserted into a compression hole in the talus and a third screw is inserted into a compression hole in the calcaneus and compression is driven by on the screws in the direction of the long axis to compress a bone interface at each of the tibiotalar joint and the talocalcaneal joint and including the step of using an associated outrigger to stabilize the nail.

15. A method of surgery as set forth in claim 14, wherein the outrigger holds the nail against rotation during the step of driving compression.

16. A method of surgery as set forth in claim 15, wherein the outrigger is used to direct the placement of a screw in a hole in the nail.
